(19) United States
(12) Patent Application Publication
(10) Pub. No.: US 2010/0037899 A1
(43) Pub. Date: Feb. 18, 2010

(54) Endotracheal tube fixation apparatus

(76) Inventors: Shiueh-Yun Lee, Taipei (TW); Yu-Li Kang, Tainan (TW)

Correspondence Address: BACON & THOMAS, PLLC, 625 SLATERS LANE, FOURTH FLOOR, ALEXANDRIA, VA, 22314-1176, US

(21) Appl. No.: 12/222,673

(22) Filed: Aug. 14, 2008

(51) Int. Cl. A61M 16/00

(52) U.S. Cl. 128/207.17

(57) ABSTRACT

The present invention relates to an endotracheal tube fixation apparatus, comprising an attracted element and a magnetic ring. The attracted element fixed on the endotracheal tube is made of magnetized substance and covered by a soft protection layer. The magnetic ring is provided with a plurality of thin-sliced magnetic elements which are spaced at intervals and enclosed by the soft protection layer. The magnetic ring is placed around the neck of a patient when in use, while the attracted element is intubated into the trachea of the patient together with the endotracheal tube and stays at the place matching correspondingly to the magnetic ring. The magnetic ring and the attracted element then attract each other, which enables a fixation of the endotracheal tube staying within a selected region.

### BACKGROUND OF THE INVENTION

1. Field of the Invention

The present invention relates to an endotracheal tube fixation apparatus, comprising an attracted element fixed on the endotracheal tube and a flexible magnetic ring; the magnetic ring is provided with a plurality of magnetic elements which can be induced to be magnetic alternatively, and is placed around the neck of a patient when in use, while the attracted element is intubated into the trachea of the patient together with the endotracheal tube and stays at the place matching correspondingly to the neck of the patient for the attraction by the magnetic elements, enabling a fixation of the endotracheal tube together with the attracted element within a selected region.

2. Description of the Prior Art

Endotracheal intubation is a procedure for patients requiring assisted breathing or lung protection. The physician operates prerequisite steps of anesthesia and dilation on the patient lying in a supine position with the mouth open, then intubates the front of the tube along the larynx and epiglottis into the trachea to a predetermined distance, and holds the tube for the subsequent treatment. The depth of the tube in the trachea is better within some range in the front portion, and the tube is usually fixed by some easy ways. The most popular way of fixation of the tube is through the patches; the invention, U.S. Pat. No. 4,270,529, which fixes the tube by a device hanging on the face of the patient. Either through a plain way or by an elaborate fixation apparatus, the use of them is subject to the fixing on the face of the patient. The patient is a life being, instead of a standstill object; therefore, any overturns of the body or any acts of scratching at the face, will easily move the tube or make the tube come off. If caretakers even don't notice that, an unpredictable accident could be happen, and this indeed deteriorates the consequence.

### SUMMARY OF THE INVENTION

In the light of the aforesaid drawbacks, the present inventor conceived the idea of the improvement to benefit the patients and accomplished the present invention due to considerable devotion and perseverance.

The objective of the present invention is to provide a fixation apparatus that immobilizes the tracheal tube within a certain range by the use of the magnetic attraction.

To accomplish the above objective, an endotracheal tube fixation apparatus of the present invention is designed, which comprises an attracted element and a magnetic ring; where the attracted element is made of magnetizable substance and annular in shape, and is attached to the endotracheal tube; the magnetic ring contains a plurality of thin-sliced magnetic elements separated at intervals, followed by the wrap of the magnetic elements by a protection layer shaped with flexible substance, to form a thin bar; during the use, the magnetic ring is placed around the neck of a patient, while the attracted element is intubated into the trachea of the patient together with the tube and stays at the place to match correspondingly to the magnetic ring, and through the attraction of the magnetic ring for the attracted element, which enables a fixation of the tube within a selected region.

In the above endotracheal tube fixation apparatus, the attracted element is further capable of being wrapped by a soft protection layer.

In the above endotracheal tube fixation apparatus, each magnetic element is affixed to an isolation pad, and the composite of the magnetic element and the isolation pad is then completely embedded in the protection layer.

In the above endotracheal tube fixation apparatus, the magnetic elements are induced by turns to magnetically attract the attracted element.

In the above endotracheal tube fixation apparatus, the side of the magnetic element opposite to the isolation pad is oriented toward the patient.

In the above endotracheal tube fixation apparatus, the magnetic ring is first placed in the fitting trough of a neck-protected pillow, followed by placing the pillow around the neck of the patient, enabling the magnetic ring to encircle the neck of the patient and to attract the attracted element that is attached to the endotracheal tube.

### BRIEF DESCRIPTION OF THE DRAWINGS

FIG. 1 is a three-dimensional external view showing the exemplified embodiment of the present invention;

FIG. 2 is a partly enlarged view of the endotracheal tube of the exemplified embodiment of the present invention;

FIG. 3 is a three-dimensional external view of the magnetic ring of the exemplified embodiment of the present invention;

FIG. 4 is a partly sectional view of the magnetic ring of the exemplified embodiment of the present invention;

FIG. 5 is a schematic diagram showing the way for the use of the exemplified embodiment of the present invention;

FIG. 6 is a schematic diagram showing the other way for the use of the exemplified embodiment of the present invention; and

FIG. 7 is a three-dimensional external view of the composite of the magnetic ring and the neck-protected pillow of the other way for the use of the exemplified embodiment of the present invention.

### DETAILED DESCRIPTION OF THE INVENTION

To achieve the aforesaid objects of the present invention, the technique adopted and the function achieved are detailed described with reference to the following preferred embodiments and the accompanying drawings, which would give a thorough comprehension on the present invention.

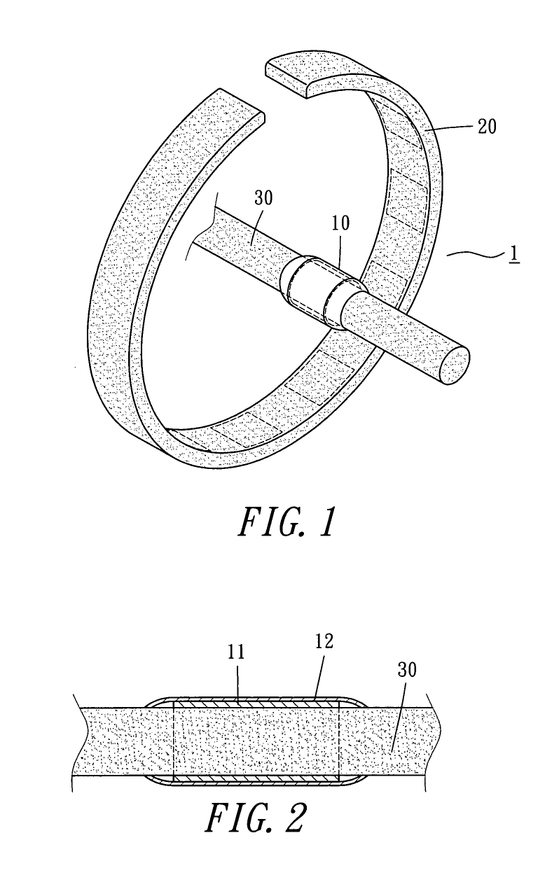

Referring to FIG. 1, the exemplified embodiment of the present invention is a fixation apparatus 1 used in the tracheal tube 30, comprising an attracted element 10 and a magnetic ring 20, where the attracted element 10 is a wrapping attached to the tracheal tube 30, where the wrapping means that the attracted element 10 encircles the tracheal tube 30 at a distance to one end of the tracheal tube 30 but without any interference on the regular ins and outs of the tracheal tube 30; referring to FIG. 2, the attracted element 10 is made of magnetizable substance 11 shaped annular to attach to the tracheal tube 30, and could be further wrapped by a soft protection layer 12; during the realization, the thickness of the attracted element 10 composed of the magnetized substance 11 and the protection layer 12 is made as small as better, which facilitates the ease of the intubation of it and accompanying tracheal tube 30 into the trachea of the patient, and the protection layer 12 is also help in avoiding any hurt to the trachea of the patient.

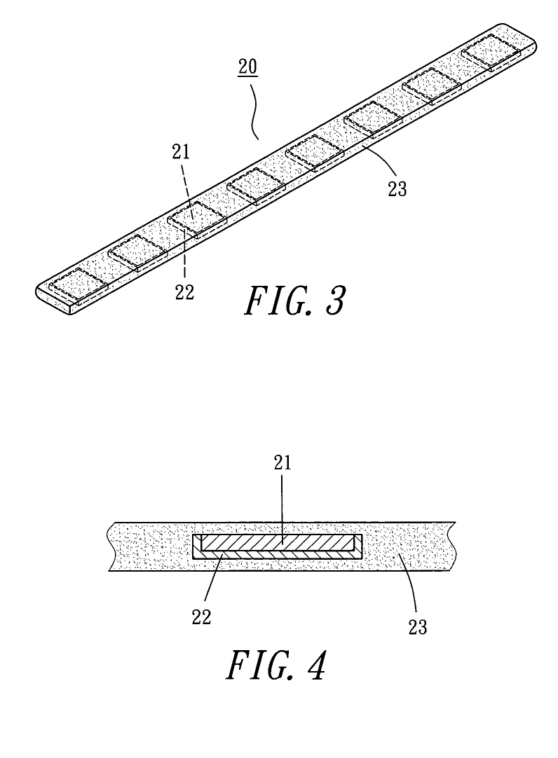

Referring to FIGS. 3-4, the structure of the magnetic ring 20 is made up of magnetic element 21, isolation pad 22 and protection layer 23, where the magnetic element 21 is a thin-sliced magnetic component where a plurality of them are separated at intervals and each magnetic element 21 has its one common side attached to an isolation pad 22, followed by the wrapping of the magnetic elements 21 and the isolation pad 22 completely by the protection layer 23 to form a thin bar (shown in FIG. 3). Each magnetic element 21 is able to be induced independently and all of the magnetic elements 21 are induced by turns (in a definite or a random order); that is, the magnetic elements 21 are not simultaneously exerting their magnetic forces but taking turns, and the alternation is not necessary to be one at a time, it could be two or more at a time, where the number and order of the alternation is not restricted, and the magnetic elements 21 induced to assert magnetic force alternatively can be controlled merely by operating the other control unit (not shown in the figure). The order of the alternation is set up into computer automatic control, and we are not going to give any description here since it is not claimed by the present invention.

The magnetic ring 20 is fabricated into a thin bar shape (shown in FIG. 3). Since the protection layer 23 and the isolation pad 22 are shaped out of flexible substances, and the embedded magnetic elements 21 are shorter pieces and spaced at intervals, the magnetic ring 20 is therefore available to bend, where it can stays in whatever shape it becomes; for instance, it is quite often bent into a ring shape (shown in FIG. 1).

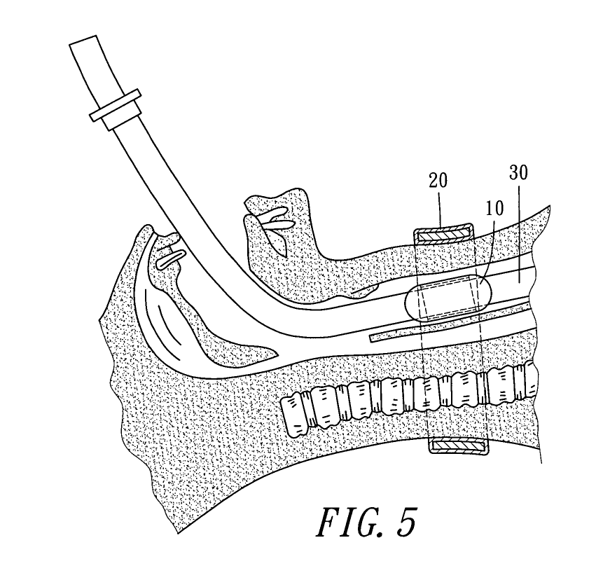

According to the aforementioned structure, the use of the present invention is shown in FIG. 5, where the magnetic ring 20 is bent to place around the neck of a patient, and the side of the embedded magnetic elements 21 opposite to the isolation pad 22 is facing inside (facing the neck of the patient). Once the tracheal tube 30 is intubated into the trachea of the patient by a surgeon to a predetermined depth for a stay, the attracted element 10 attached to the tracheal tube 30 will match correspondingly the magnetic ring 20 around the neck of the patient; this is because the surgeon can tell the depth and location from the prior scan or irradiation, and the adjustment for the depth can be either by shifting a distance for the tracheal tube 30 which has the attracted element 10 attached to it, or by shifting the attracted element 10 a distance directly on the tracheal tube 30 followed by the intubation, and these are something which are firm before the surgeon implementing the tracheal intubation.

Subsequently, the control unit (not shown in the figure) is taking control of the electricity conduction to the magnetic elements 21 in the magnetic ring 20 by turns, and having the magnetic elements 21 induced alternatively, where the induced magnetic force attracts inwardly the magnetized substance 11 in the attracted element 10, that is, attracting the attracted element 10, which enables the tracheal tube 30 to stay within a certain range.

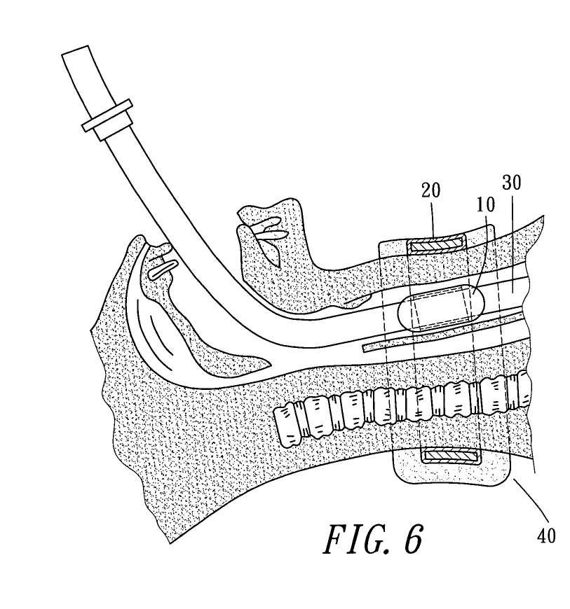

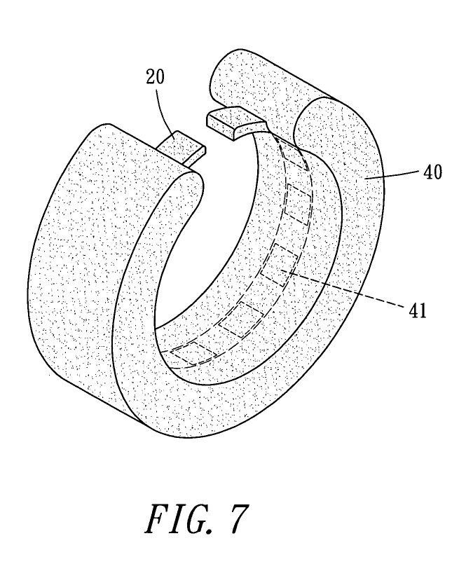

The way of use shown in FIG. 5 shows that the magnetic ring 20 encircles the neck of a patient; a more comfortable way for the neck of a patient that achieves the same effectiveness is shown in FIGS. 6-8, which is done by placing the magnetic ring 20 in a fitting trough 41 of the neck-protected pillow 40, followed by placing the neck-protected pillow 40 around the neck of a patient, and the magnetic ring 20 as above encircles the neck of the patient and attracts the attracted element 10 on the tracheal tube 30. In this way of the use, the additional neck-protected pillow 40 is for the support of the neck of the patient, which attracts the attracted element 10 the same way but no doubt makes the patient much more comfortable.

The tracheal tube fixation apparatus 1 of the present invention has the following advantages when in use:
1. Since the magnet ring 20 is a thin piece around the neck of a patient, instead of fixing at the face of the patient, the patient won't feel any uncomfortable at all.
2. Since the magnet ring 20 magnetically attracts the attracted element 10 in a physically untouched way to each other, the patient won't feel any physical pressures on the neck.
3. The magnetic elements 21 embedded in the magnet ring 20 are induced alternatively to attract the attracted element 10 in a ring configuration, which avoids a sustained pressure only on the same part of the trachea.
4. The magnetic element 21 embedded in the magnet ring 20 is attaching to the isolation pad 22, where its magnetic field is axial oriented, and this arrangement could attract the attracted element 10 with only a moderate magnetic force.

In conclusion, the present invention indeed accomplishes expected objective and function, and the disclosure of its structure and composition has not opened to the public, which is construed to be creative and novel; moreover, the application of the present invention is ample enough for those skilled in the art to realize it, which is compliant to the disclosure requirements; an application is now for filing according to the law, which deserves your favorable examination and approval.

What is claimed is:

1. An endotracheal tube fixation apparatus, comprising an attracted element and a magnetic ring; where said attracted element is made of magnetizable substance and annular in shape, and is attached to the endotracheal tube; said magnetic ring containing a plurality of thin-sliced magnetic elements separated at intervals, followed by the wrapping of the magnetic elements by a protection layer shaped out of flexible substance to form a thin bar; during the use, said magnetic ring being placed around the neck of a patient, while said attracted element is intubated into the trachea of the patient together with the endotracheal tube and stayed at the place that is matching correspondingly to said magnetic ring, and through the attraction of said magnetic ring for said attracted element, which enables a fixation of the endotracheal tube within a selected region.

2. An endotracheal tube fixation apparatus as in claim 1 wherein the attracted element is further capable of being wrapped by a soft protection layer.

3. An endotracheal tube fixation apparatus as in claim 1 wherein each magnetic element has its one common side affixed to an isolation pad, and the composite of the magnetic element and the isolation pad being then completely embedded in the protection layer.

4. An endotracheal tube fixation apparatus as in claim 1 wherein the magnetic elements are induced alternatively to magnetically attract said attracted element.

5. An endotracheal tube fixation apparatus as in claim 3 wherein the side of the magnetic elements opposite to the isolation pad is oriented toward the patient.

6. An endotracheal tube fixation apparatus as in claim 1 wherein said magnetic ring is first placed in the fitting trough of a neck-protected pillow, followed by placing the neck-protected pillow around the neck of the patient, enabling said magnetic ring to encircle the neck of the patient and to attract said attracted element that is attached to the endotracheal tube.
